(19) United States
(12) Patent Application Publication
(10) Pub. No.: US 2011/0036133 A1
(43) Pub. Date: Feb. 17, 2011

(54) PURIFICATION OF PROTEIN HYDROLYSATE AND THE RESULT AND PRODUCTS

(75) Inventors: Timothy James Hadden, Sloan, IA (US); Gary Merle Kurtz, Correctionville, IA (US)

Correspondence Address: ORGANON USA, INC.;c/o MERCK, 2000 Galloping Hill Road, Mail Stop: K-6-1, 1990, Kenilworth, NJ, 07033, US

(73) Assignee: N. V. ORGANON

(21) Appl. No.: 12/988,180

(22) PCT Filed: Apr. 15, 2009
(86) PCT No.: PCT/EP09/54567

§ 371 Date: Oct. 15, 2010

Related U.S. Application Data
(60) Provisional application No. 61/046,206, filed on Apr. 18, 2008.

(30) Foreign Application Priority Data
Apr. 24, 2008 (EP) 08155067.5

(51) Int. Cl. C07K 2/00; C07K 1/34; A23J 1/00; C05F 11/00

(52) U.S. Cl. 71/15; 530/344; 530/300; 426/656

(57) ABSTRACT

The present invention relates to a process to purify enzymatically digested heparin-derived protein hydrolysate (peptone) comprising the step of passing the peptone through a nanofilter at a temperature of about ambient to about 130° F. and a pressure of about ambient to about 360 psi resulting in peptone concentrate.

This invention relates to a process to purify protein hydrolysate (peptone). This invention further relates to the resulting purified protein hydrolysate (peptone concentrate) and its use in food and fertilizer.

### BACKGROUND OF THE INVENTION

Protein hydrolysate comprises a mixture which includes amino acids and short chain peptides resulting from the hydrolysis of various animal and vegetable proteins. For example, protein hydrolysates are common by-products of the extraction of the blood anti-coagulant heparin from porcine hash gut or intestinal mucosa.

For both economic and environmental reasons, productive use is now being made of an increasing percentage of the waste material generated as a result of the slaughter of animals, such as livestock. A major use of livestock waste or other by-products is in the production of the blood anti-coagulant heparin. The small intestine raw material is collected in slaughterhouses and preserved by stirring in a preservative, typically sodium metabisulfite or liquid sodium bisulfite. Sodium bisulfite is the industry standard for preserving heparin bearing raw materials, although other preservatives such as phosphoric acid, lactic acid, or various peroxides have been tested and found to be at least somewhat effective, albeit cost prohibitive. The heparin process involves increasing the pH of the raw material to alkaline, adding a proteolytic enzyme to digest the material, separating the fat constituents of the solution by acid or base addition, if necessary, and removing the heparin from the resulting aqueous solution using an ion exchange resin. After the ion exchange resin has adsorbed the heparin and then been collected by screening, the remaining aqueous liquid hydrolysate (also referred to in the art as peptone) has historically been used as a fertilizer or a feed additive.

Purified protein products from protein hydrolysate may have a multitude of potential uses such as cosmetic additives, nutritional ingredients for foods and beverages, foaming agents, additives to medicinal compounds to block bitterness, sources of amino acids, additives or replacements for infant formula, and use in artificial nutrition administered orally, internally, parenterally or intravenously.

Of particular interest in the present invention is the use of purified protein hydrolysate as a feed ingredient for livestock. Nutritional uses of the purified protein hydrolysate also include such specialty feeds as milk replacers for calf, piglet and other weaning mammals, protein extender for animal feed, an amino acid supplement, and flavor or protein enhancer for human food and pet food.

A particular problem with use of this material as a feed additive has been the presence of the preservative, which is concentrated as water is removed from the peptone during drying operations which employ evaporation. For example, by reducing water content from approximately 82% (as is found in many commercially available protein hydrolysates available as by-products from the production of heparin) to 55% or less water content the sodium sulfite levels are also being concentrated. For example, a typical level of sulfite in an 18% solid by weight liquid protein hydrolysate is 2.5% to 3.5%. However, when this same 18% solid by weight protein hydrolysate is concentrated through evaporation of the water to a water content of 55% the sulfite concentration is increased to 6.25% to 8.75% in the concentrated product. This level of sulfite is found to be undesirable by many end product users and completely unacceptable by many more. For example, the protein hydrolysates as potential sources of nutrient become unpalatable with the presence of high sulfite levels when used in the pet food market. Further, the Association of American Feed Control Officials (AAFCO) (official publication at pages 196-197) restricts the use of sulfites in meats and vitamin BI sources. Moreover this treatment requires a considerable amount of energy. There have been attempts to remove the preservative by treatment with a chemical compound and precipitation of the preservative salts, but these methods were inefficient and thus cost prohibitive (U.S. Pat. No. 5,607,840 and U.S. Pat. No. 6,051,687).

One method of reducing the salt level is by membrane filtration as disclosed in U.S. Pat. No. 6,051,687. One material was an 18% solid by weight liquid protein hydrolysate as received from the heparin extraction source and the other was a low fat material produced from the 18% solid by weight liquid protein hydrolysate. The low fat material was used as there was a concern that the fatty components would interfere with the membrane filtration. However, little difference was noted between the two materials. The study showed that the concept could work albeit with some disadvantages such as the loss of 10% of the crude protein which could potentially be solved by the use of different type of membranes. However, no further testing was reported.

Despite an increased interest in alternate uses for the protein hydrolysate by-product of heparin extraction from animal tissue, it has not been previously known how to reduce the salt concentrations in the protein hydrolysate in an efficient manner. The present invention provides a method for purifying the protein hydrolysate. When the hydrolysate is concentrated, it results in a protein product with significantly reduced salt concentration. In addition, it has not been known how to remove the sulfites or sulfates from this protein hydrolysate so that the hydrolysate in an efficient manner, when concentrated, results in a significantly reduced sulfite and sulfate concentrations.

### SUMMARY OF THE INVENTION

The present invention relates to a process to purify enzymatically digested heparin-derived protein hydrolysate (peptone) comprising the step of passing the peptone through a nanofilter at a temperature of about ambient to about 130° F. and a pressure of about ambient to about 360 psi resulting in peptone concentrate.

The problem of concentrating the preservative with the removal of the water from the peptone in an energy efficient manner is solved by nanofiltration of the peptone to remove both water and a large portion of the preservative salts. By employing nanofiltration contrary to other methods previously tested, the bulk of the preservative is removed and the nutrient quality of the peptone is maintained at a high level. In addition, it allows the factory to maintain stable product throughout the production process.

Once the preservative is removed, the concentrated peptone can be used as is, concentrated further, diluted with water and concentrated again, or combined with previously separated fractions of the peptone. Once the desired final product has been achieved, i.e. the purified enzymatically digested heparin-derived protein hydrolysate, it can be used as a livestock feed or feed additive/supplement, a pet food additive/supplement, as a human or animal additive/supplement, or a fertilizer or fertilizer concentrate. In this respect waste generation will be minimized.

### FIGURES

FIG. 1 shows an example of a flow scheme to purify protein hydrolysate from porcine small intestine in the heparin extraction process according to the present invention.

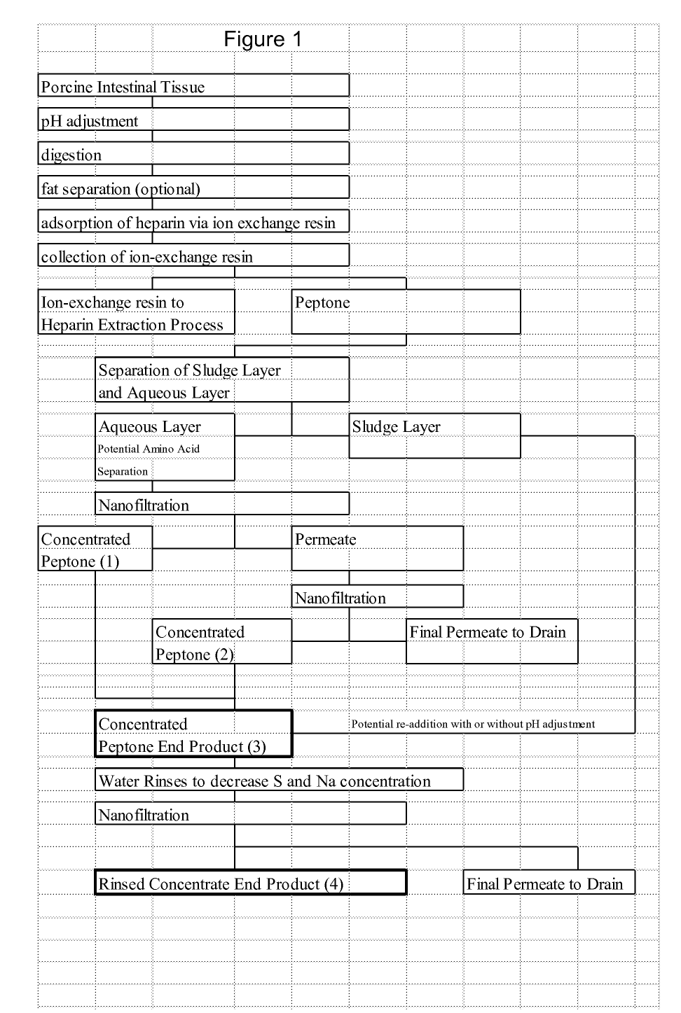

### DETAILED DESCRIPTION

The present process comprises the step of passing the peptone through a nanofilter at a temperature of about ambient to about 130° F. and a pressure of about ambient to about 360 psi resulting in peptone concentrate.

The peptone may be commercially available liquid protein hydrolysate by-product from the extraction of heparin from porcine hash gut or intestinal mucosa. The process can also be carried out in the factory where the heparin extraction takes place. More particularly, starting with the digested porcine small intestine feedstock of the heparin extraction process, the heparin is extracted from the solution by using an ion exchange resin. The ion exchange resin is sieved from the peptone and the peptone is collected for further treatment. The peptone is essentially heparin free.

Peptone can be initially nanofiltered to remove a portion of the preservative and the water. This peptone concentrate can then subsequently be evaporated to the desired moisture. Nanofiltration to remove a portion of the preservative and the water is more economical than evaporation alone. However, at a certain moisture level the nanofilters start to blind and efficiency is lost making evaporation for the final concentration more efficient and necessary.

An alternative embodiment of the process of the present invention comprises the steps of

a) acidifying the peptone to a pH of about 4 to about 7 obtaining a sludge layer comprising fatty and/or flocculated components and an aqueous layer,
b) separating the sludge layer from the aqueous layer,
c) passing the aqueous layer through a nanofilter membrane at a temperature of about ambient to about 130° F. and a pressure of about ambient to about 360 psi resulting in peptone concentrate (1).

The peptone is acidified to a pH of about 4 to about 7 obtaining a sludge layer comprising fatty and/or flocculated components and an aqueous layer. Acidification is known to the skilled man. Use can be made of inorganic acids. Examples of inorganic acid include hydrochloric acid, phosphoric acid, sulfuric acid, or nitric acid. Hydrochloric acid is preferred.

The sludge layer is separated from the aqueous layer, preferably by decanting.

The sludge containing peptone or the aqueous layer (the clear peptone fraction) is passed at a relatively low temperature in the range of about ambient to about 150 degrees F. (20 to 65° C.), preferably about 90 to about 130 F (30 to 55° C.) through a nanofiltration membrane at varying pressures between about ambient to about 360 psi (1 to 25 bar) preferably about 100 to about 300 psi (7 to 20 bar), but typically at about 250 to about 280 psi (17 to 19 bar) resulting in peptone concentrate.

The nanofiltration membrane used in the present invention may be selected from polymeric and inorganic membranes. Pore size of these nanofiltration membranes allows a molecular weight cut off of 150-300 Daltons.

Typical polymeric nanofiltration membranes useful in the present invention include, for example, polyether sulfone membranes, sulfonated polyether sulfone membranes, polyester membranes, polysulfone membranes, aromatic polyamide membranes, polyvinyl alcohol membranes and polypiperazine membranes and combinations thereof. Cellulose acetate membranes are also useful as nanofiltration membranes in the present invention.

Typical inorganic membranes include ZrO₂— and Al₂O₃-membranes, for example.

Preferred nanofiltration membranes are selected from sulfonated polysulfone membranes and polypiperazine membranes. For example, useful membranes are Desal D series manufactured by GE Osmonics/General Electric Co. Water technologies such as models within the DL series, such as DL 4040 and DL 2540. The nanofiltration membranes which are useful in the present invention may have a negative or positive charge. The membranes may be ionic membranes, i.e. they may contain cationic or anionic groups, but even neutral membranes are useful. The nanofiltration membranes may be selected from hydrophobic and hydrophilic membranes.

The typical form of nanofiltration membranes is a flat sheet form. The membrane configuration may also be selected e.g. from tubes, spiral wound membranes and hollow fibers. “High shear” membranes, such as vibrating membranes and rotating membranes can also be used.

Before the nanofiltration procedure, the nanofiltration membranes may be pretreated by washing with a washing agent, typically with an acidic washing agent. Also alkaline washing agents or alcohols may be used.

The permeate from the process of the present invention will contain water and various salts, along with a portion of the preservative salt, such as the sodium bisulfite plus some amino acids.

In an alternative embodiment of the present invention, the peptone concentrate (1) from the process of the present invention is recirculated repeatedly through the nanofilter until the desired concentration is reached or until the permeate flow stops, typically at or below a water concentration of approximately 70-75%.

In a preferred embodiment, the permeate is passed through an additional nanofilter which can be the same as used in the first nanofiltration step as mentioned above resulting in peptone concentrate (2). Alternatively, a membrane having a pore size allowing a molecular weight cut off of <150 Daltons using, for example, GE Osmonics model DK series of finer membrane pore size to concentrate the amino acids that had been transferred into the original permeate. The concentrated amino acids from the permeate (peptone concentrate (2)) can then be mixed in with the initial concentrate (peptone concentrate (1)) to obtain peptone concentrate (3).

In another embodiment, soft water rinse(s) may be added to the peptone concentrate (1) or (3) and nanofiltration may be resumed, allowing further removal of various minerals resulting in peptone concentrate (4).

Optionally, depending on the fat content of the raw material used in the heparin extraction process, fat may be removed. Fat removal is achieved at a pH of about 6 to about 9 at a temperature of about 130 to about 160° F. The fatty components tend to flocculate and rapidly float to the top. The fatty components can be removed by a number of methods including separation by centrifuging, decanting, or filtering. The optimum pH level for separation varies according to the method used. The heparin is extracted either before or after fat separation using an ion exchange resin.

Optionally, individual or groups of amino acids can be separated from the protein hydrolyzate and isolated using various known techniques such as but not limited to precipitation, ion exchange, chemical catalyst reaction, or additional filtration.

The concentrated peptone (1), (3), or (4) typically has a pH of about 5.5 and can be stored indefinitely, due to its pH and lower water concentration/activity. The pH may be adjusted before, during, or after concentration and/or combination of this fraction with other materials such as the sludge that had been optionally removed prior to nanofiltration. A preferred embodiment is a purified protein hydrolysate comprising the original concentrate (peptone concentrate (1)), the concentrate fraction from the nanofiltration of the original permeate (peptone concentrate (2)), and fatty and/or flocculated components. These fatty and/or flocculated components may be obtainable from the sludge layer or the fat removal step. The purified protein hydrolysate may also be peptone concentrate (3) or (4), optionally in combination with peptone concentrate (2), optionally in combination with fatty and/or flocculated components.

A product according to the present invention is a purified enzymatically digested heparin-derived protein hydrolysate comprising

1) less than 12,000 ppm sulfur
2) less than 21,000 ppm sodium
3) more than 120,000 ppm amino acid
4) 80% moisture or less
having a sulfur to total nitrogen ratio of 0.5 or less and a sodium to total nitrogen ratio of 1.0 or less.

Preferably, the purified protein hydrolysate has a sulfur to total nitrogen ratio less than 0.4. Alternatively, the sodium to total nitrogen ratio may be less than 0.9, preferably less than 0.8, more preferably less than 0.7, most preferably less than 0.6.

More preferably, the purified protein hydrolysate comprises fatty and/or flocculated components.

The following examples involve experiments conducted which demonstrate how the above-stated embodiments were reached. They are not meant to limit the present invention in any manner. All references to patents, journal articles or other publications cited previously or hereinafter are hereby expressly incorporated in their entirety by reference.

Further it is understood that trivial modifications would be known to those of skill in the art and are also understood to be included within the scope of the present invention as described and claimed herein.

### Example 1 and Comparative Example A

A batch of peptone was taken from the heparin extraction process. The peptone was acidified and the sludge was removed therefrom resulting in Aqueous Starting Material. The Aqueous Starting Material was passed through a nanofilter, i.e. GE Osmonics model DL 4040 membrane, at a nominal pH of 5.5, a temperature of 100-120 degree F., and a nominal pressure of 270 psi.

The results are listed in Table 1 (Membrane Filtration Run).

For comparison's sake, a batch of peptone was separated as in the Membrane Filtration Run. The water layer was evaporated to see the effect on the relative concentration of sodium and sulfur. At a concentration of 2.13, this solution became highly viscous and the evaporation run was stopped.

Results are also listed in Table 1 (Evaporation Run).

TABLE 1

| | | Example 1 | | | | | | |
|---|---|---|---|---|---|---|---|---|
| | | Membrane Filtration Run | | | | Comparative Example A | | |
| | | | | | Starting | Evaporation Run | | |
| | | | | | Material | | | Evaporated |
| | | | | | Calculated | | | Water Layer |
| | | Aqueous | | | to | Aqueous | Evaporated | Calculated |
| | | Starting | | | Equivalent | Starting | Water | to 4.00 X |
| Parameter | Units | Material | Permeate | Concentrate | Protein | Material | Layer | concentration |
| Volume | Gal | 2400 | 1800 | 600 | NA | NA | NA | NA |
| Sampled | | | | | | | | |
| Conc. Factor | | 1.00 | | 4.00 | | | 2.13x | 4.00x |
| Ammonium N | ppm | 908 | 840 | 1789 | 1581 | 1200 | 6800 | 4320 |
| Organic N | ppm | 13671 | 7045 | 23588 | 23796 | 14900 | 25100 | 47135 |
| Total N | ppm | 14579 | 7885 | 25377 | 25377 | 16100 | 31900 | 59905 |
| Phosphorus | ppm | 2961 | 1942 | 5425 | 5154 | 3000 | 6700 | 12582 |
| P2O5 | | | | | | | | |
| Potassium | ppm | 2454 | 2585 | 2921 | 4272 | 2200 | 5000 | 9390 |
| K2O | | | | | | | | |
| Sulfur | ppm | 8225.0 | 5420.0 | 11797 | 14317 | 8200 | 16300 | 30610 |
| Calcium | ppm | 85.0 | 53.8 | 180 | 148 | 100 | 200 | 376 |
| Magnesium | ppm | 121.5 | 80.2 | 231 | 211 | 100 | 300 | 563 |
| Sodium | ppm | 19969.0 | 20198 | 17353 | 34759 | 14900 | 36900 | 69295 |
| Copper | ppm | 1.1 | Nd | 2.6 | 1.91 | 1 | 2 | 3.8 |
| Iron | ppm | 16.6 | 2.8 | 41.2 | 28.9 | 17 | 37 | 69.5 |
| Manganese | ppm | 1.2 | 0.6 | nd | 2.09 | 1 | 3 | 5.6 |
| Zinc | ppm | 11.7 | 0.6 | 32.3 | 20.4 | 12 | 28 | 52.6 |
| pH | na | 5.55 | 5.47 | 5.7 | NA | 5.2 | 5.8 | 5.7 |
| Moisture | % | 82.20 | NA | 74.53 | 69.0 | 83.1 | 63.8 | 32.0 |
| Solids | % | 17.80 | NA | 25.47 | 31.0 | 16.9 | 36.2 | 68.0 |
| Amino Acids | | | | | | | | |
| Alanine | % | 0.75 | 0.36 | 0.90 | 1.31 | 0.88 | 2.03 | 3.81 |
| Arganine | % | 0.44 | 0.20 | 0.81 | 0.77 | 0.46 | 0.75 | 1.41 |
| Aspartic Acid | % | 0.87 | 0.20 | 1.64 | 1.51 | 0.81 | 1.89 | 3.55 |
| Cystine | % | 0.09 | 0.09 | 0.33 | 0.16 | 0.19 | 0.34 | 0.64 |
| Glutamic Acid | % | 1.34 | 0.43 | 2.60 | 2.33 | 1.31 | 2.83 | 5.31 |
| Glycine | % | 0.82 | 0.35 | 1.58 | 1.43 | 0.78 | 1.73 | 3.25 |
| Histidine | % | 0.20 | 0.11 | 0.35 | 0.35 | 0.18 | 0.40 | 0.75 |
| Isoleucine | % | 0.34 | 0.21 | 0.49 | 0.59 | 0.33 | 0.76 | 1.43 |
| Leucine | % | 0.67 | 0.44 | 0.94 | 1.17 | 0.66 | 1.49 | 2.80 |
| Total Lycine | % | 0.67 | 0.31 | 1.25 | 1.17 | 0.65 | 1.41 | 2.65 |
| Methionine | % | 0.14 | 0.10 | 0.18 | 0.24 | 0.15 | 0.31 | 0.58 |
| Phenylalinine | % | 0.36 | 0.31 | 0.50 | 0.63 | 0.39 | 0.78 | 1.46 |
| Proline | % | 0.58 | 0.28 | 0.97 | 1.01 | 0.57 | 1.20 | 2.25 |
| Serine | % | 0.50 | 0.24 | 0.62 | 0.87 | 0.35 | 0.80 | 1.50 |
| Threonine | % | 0.49 | 0.24 | 0.67 | 0.85 | 0.42 | 0.98 | 1.84 |
| Tryptophan | % | 0.22 | NA | 0.14 | 0.38 | 0.13 | 0.21 | 0.39 |
| Tyrosine | % | 0.32 | 0.21 | 0.45 | 0.56 | 0.32 | 0.63 | 1.18 |
| Valine | % | 0.42 | 0.29 | 0.69 | 0.73 | 0.43 | 1.08 | 2.03 |
| S:N ratio | | 0.56 | 0.69 | 0.465 | | 0.51 | 0.51 | |
| Na:N ratio | | 1.37 | 2.56 | 0.68 | | 0.93 | 1.15 | |
| Amino acid | ppm | 92200 | 43700 | 151100 | | 90100 | 196200 | |

nd = not detected

### Evaluation of Results

When comparing the Concentrate prepared according to the present invention to the Aqueous Starting Material prior to nanofiltration, we see the following:
Decrease in volume by 4.00×
Increase in total N by 1.74×
Increase in Sulfur concentration by 1.43×
Decrease in Sodium concentration by 0.87×
Increase in concentration of most Amino Acids about 1.5−2×

A sodium and sulfur concentration increase of 4.00× would be expected if the sodium bisulfite preservative had stayed in the concentrate.

The Permeate from the nanofiltration step smelled highly of sodium bisulfite preservative, which is supported by the high concentration of sodium and sulfur in the results for the Permeate in Table 1.

The resultant removal of the sodium bisulfite from the protein hydrolyzate makes this a much more palatable feed additive. The transfer of some of the amino acids into the initial permeate is acceptable in light of the removal of the bulk of the sodium bisulfite and the potential mechanism of a secondary nanofiltration of the permeate to capture the bulk of the amino acids that had been transferred to the permeate. Also, the Concentrate according to the invention has a low comparative viscosity, making it easy to handle in comparison to the evaporated material.

Finally, it is understood that with soft water rinse(s) added to the Concentrate nanofiltration may be resumed, allowing further removal of various minerals, which may result in a sulfur to total nitrogen ratio less than 0.4 and a sodium to total nitrogen ratio less than 0.9.

The addition of the sludge removed prior to the nanofiltration to the Concentrate would provide a purified protein hydrolysate comprising fatty/flocculated components.

The evaporation run, which is the comparative example, resulted in a highly viscous solution at a concentration of 2.13. This material was tested and the results were converted to theoretical results that would have been seen had we been able to continue the evaporation to a level of 4.00. It can be seen from the results from Table 1 that the sodium and sulfur concentration levels were maintained in the evaporated material and were not lost during evaporation.

### Example 2

Another batch of peptone MW2 from the heparin extraction process was acidified to remove the sludge MW4 therefrom. The aqueous starting material MW5 was passed through a nanofilter, i.e. DL 2540, to result in peptone concentrate (1) MW7. The permeate therefrom was passed again through the same nanofilter providing concentrate (2) MW10. The two peptone concentrates (1) and (2) (MW7+MW10) and the sludge MW4 previously removed were added together resulting in a purified protein hydrolysate comprising fatty/flocculated components MW14 (peptone concentrate (3)).

Results are listed in Table 2.

MW7 and MW14 are purified protein hydrolysates according to the present invention.

TABLE 2

| Parameter | Units | MW2 | MW4 | MW5 | MW7 | MW10 | MW14 |
|---|---|---|---|---|---|---|---|
| Ammonium N | ppm | 800 | 600 | 800 | 600 | 800 | 1100 |
| Organic N | ppm | 16500 | 17000 | 15400 | 23900 | 16400 | 21000 |
| Total N | ppm | 17300 | 17600 | 16200 | 24500 | 17200 | 22100 |
| Phosphorus P2O5 | ppm | 3700 | 7500 | 2700 | 4200 | 3400 | 5500 |
| Potassium K2O | ppm | 2700 | 2200 | 2300 | 1900 | 2300 | 2100 |
| Sulfur | ppm | 10800 | 7400 | 8900 | 8800 | 9700 | 8300 |
| Calcium | ppm | 200 | 600 | 100 | 200 | 100 | 300 |
| Magnesium | ppm | 200 | 300 | 100 | 200 | 200 | 200 |
| Sodium | ppm | 18600 | 14800 | 15400 | 13500 | 16100 | 20300 |
| Copper | ppm | 2 | 9 | 1 | 4 | 0 | 6 |
| Iron | ppm | 31 | 86 | 17 | 63 | 18 | 85 |
| Manganese | ppm | 2 | 7 | 1 | 3 | 2 | 4 |
| Zinc | ppm | 19 | 18 | 13 | 47 | 6 | 35 |
| pH | na | 8.4 | 5.3 | 6.1 | 7.6 | 5.6 | 7.2 |
| Moisture | % | 81.1 | 48.5 | 84.6 | 79.7 | 83.8 | 66.8 |
| Solids | % | 18.9 | 51.5 | 15.4 | 20.3 | 16.2 | 33.2 |
| Amino Acids | | | | | | | |
| Alanine | % | 0.64 | 0.51 | 0.46 | 0.97 | 0.46 | 0.88 |
| Arganine | % | 0.45 | 0.61 | 0.67 | 1.95 | 1.14 | 1.18 |
| Aspartic Acid | % | 0.69 | 0.62 | 0.76 | 1.43 | 0.84 | 0.79 |
| Cystine | % | 0.20 | 0.20 | 0.46 | 0.38 | 0.27 | 0.34 |
| Glutamic Acid | % | 1.19 | 0.95 | 1.15 | 2.06 | 1.31 | 1.58 |
| Glycine | % | 0.86 | 0.59 | 0.79 | 1.42 | 0.51 | 1.14 |
| Histidine | % | 0.22 | 0.48 | 0.21 | 0.39 | 0.32 | 0.26 |
| Isoleucine | % | 0.39 | 0.52 | 0.26 | 0.32 | 0.38 | 0.63 |
| Leucine | % | 0.77 | 1.02 | 0.59 | 0.60 | 0.47 | 0.99 |
| Total Lycine | % | 0.63 | 0.83 | 0.59 | 1.02 | 0.91 | 0.91 |
| Methionine | % | 0.13 | 0.22 | 0.13 | 0.15 | 0.16 | 0.15 |
| Phenylalinine | % | 0.28 | 0.71 | 0.32 | 0.32 | 0.19 | 0.59 |
| Proline | % | 0.52 | 0.52 | 0.58 | 0.93 | 0.41 | 0.65 |
| Serine | % | 0.37 | 0.37 | 0.41 | 0.49 | 0.36 | 0.40 |
| Threonine | % | 0.38 | 0.33 | 0.43 | 0.54 | 0.42 | 0.59 |
| Tryptophan | % | 0.12 | 0.18 | 0.13 | 0.11 | n.d. | 0.13 |
| Tyrosine | % | 0.3 | 0.56 | 0.23 | 0.29 | 0.34 | 0.52 |
| Valine | % | 0.57 | 0.78 | 0.48 | 0.58 | 0.65 | 0.35 |
| S:N ratio | | 0.62 | 0.42 | 0.55 | 0.36 | 0.56 | 0.38 |
| Na:N ratio | | 1.08 | 0.84 | 0.95 | 0.55 | 0.94 | 0.92 |
| Amino acid | ppm | 87100 | 100000 | 86500 | 139500 | 91400 | 120800 |

nd = not detected

### Examples 3 and 4

Two samples of peptone (without acidifying and no physical separation) have been nanofiltered to remove a portion of the preservative and the water.

Results are listed in Table 3.

TABLE 3

| | | Starting | | Starting | |
|---|---|---|---|---|---|
| | | material | | material | |
| Parameter | Units | for Ex. 3 | Ex. 3 | for Ex. 4 | Ex. 4 |
| Ammonium N | ppm | 2100 | 1300 | 1800 | 1900 |
| Organic N | ppm | 9300 | 23700 | 9100 | 21500 |
| Total N | ppm | 11400 | 25000 | 10900 | 23400 |
| Phosphorus P2O5 | ppm | 3200 | 11100 | 3500 | 11000 |
| Potassium K2O | ppm | 2100 | 4000 | 2400 | 4200 |
| Sulfur | ppm | 10800 | 18200 | 11700 | 19400 |
| Calcium | ppm | 200 | 500 | 200 | 600 |
| Magnesium | ppm | 100 | 400 | 100 | 400 |
| Sodium | ppm | 17300 | 33500 | 20000 | 36000 |
| Copper | ppm | 2 | 5 | 2 | 5 |
| Iron | ppm | 18 | 59 | 19 | 62 |
| Manganese | ppm | 2 | 4 | 1 | 3 |
| Zinc | ppm | 12 | 43 | 14 | 45 |
| pH | na | 8.0 | 8.3 | 7.8 | 8 |
| Moisture | % | 85.3 | 66 | 85.9 | 65.4 |
| Solids | % | 14.7 | 34 | 14.1 | 34.6 |
| Amino Acids | | | | | |
| Alanine | % | 0.51 | 0.88 | 0.58 | 0.88 |
| Arganine | % | 0.18 | 1.09 | 0.15 | 0.75 |
| Aspartic Acid | % | 0.19 | 1.45 | 0.34 | 1.44 |
| Cystine | % | 0.09 | 0.27 | 0.08 | 0.31 |
| Glutamic Acid | % | 0.87 | 2.16 | 0.96 | 2.28 |
| Glycine | % | 0.49 | 0.95 | 0.57 | 1.04 |
| Histidine | % | 0.16 | 0.37 | 0.28 | 0.33 |
| Isoleucine | % | 0.28 | 0.63 | 0.29 | 0.49 |
| Leucine | % | 0.5 | 1.05 | 0.57 | 0.86 |
| Total Lycine | % | 0.51 | 1.38 | 0.59 | 1.26 |
| Methionine | % | 0.16 | 0.33 | 0.17 | 0.24 |
| Phenylalinine | % | 0.27 | 0.51 | 0.31 | 0.46 |
| Proline | % | 0.4 | 0.95 | 0.47 | 0.92 |
| Serine | % | 0.09 | 0.35 | 0.1 | 0.32 |
| Threonine | % | 0.29 | 0.64 | 0.28 | 0.54 |
| Tryptophan | % | 0.07 | 0.34 | 0.08 | 0.17 |
| Tyrosine | % | 0.10 | 0.52 | 0.15 | 0.39 |
| Valine | % | 0.37 | 0.76 | 0.43 | 0.60 |
| S:N ratio | | 0.95 | 0.73 | 1.07 | 0.83 |
| Na:N ratio | | 1.52 | 1.34 | 1.83 | 1.54 |
| Amino acid | ppm | 55300 | 146300 | 64000 | 132800 |

nd = not detected

The results are in line with the findings of Example 1.

Furthermore, it is surprising that with the presence of sludge nanofiltration is still possible with good results without blinding the filter.

1. A process to purify enzymatically digested heparin-derived protein hydrolysate (peptone) comprising the step of passing the peptone through a nanofilter at a temperature of about ambient to about 130 F and a pressure of about ambient to about 360 psi resulting in peptone concentrate.

2. A process according to claim 1 comprising a subsequent step of evaporating the peptone concentrate to the desired moisture percentage.

3. A process according to claim 1 comprising the steps of a) acidifying the peptone to a pH of about 4 to about 7 obtaining a sludge layer comprising fatty and/or flocculated components and an aqueous layer, b) separating the sludge layer from the aqueous layer, c) passing the aqueous layer through a nanofilter at a temperature of about ambient to about 130 F and a pressure of about ambient to about 360 psi resulting in peptone concentrate (1).

4. A process according to claim 1 wherein the nanofilter has a pore size allowing for a molecular weight cut off of 150-300 Daltons.

5. A process according to claim 1 wherein in a subsequent step a permeate is recovered from the nanofiltration step and passed again through a nanofilter resulting in peptone concentrate (2).

6. A process according to claim 5 wherein the nanofilter has a pore size allowing a molecular weight cut off of <150 Daltons.

7. A process according to claim 1 wherein soft water is added to peptone concentrate

8. A process according to claims 5 wherein peptone concentrate, e.g. peptone concentrate (1), is combined with peptone concentrate (2) resulting in peptone concentrate (3), soft water is added to peptone concentrate (3) and the resulting composition is passed again through a nanofilter resulting in peptone concentrate (4).

9. A purified enzymatically digested heparin-derived protein hydrolysate obtainable by the process according to claim comprising peptone concentrate.

10. A purified protein hydrolysate according to claim 9 comprising additionally fatty and/or flocculated components.

11. Purified enzymatically digested heparin-derived protein hydrolysate comprising
1) less than 12,000 ppm sulfur
2) less than 2 1,000 ppm sodium
3) more than 120,000 ppm amino acid
4) 80% moisture or less having a sulfur to total nitrogen ratio of 0.5 or less and a sodium to total nitrogen ratio of 1.0 or less.

12. A purified protein hydrolysate according to claim 11 wherein the sulfur to total nitrogen ratio is less than 0.4.

13. A purified protein hydrolysate according to claim 11 wherein the sodium to total nitrogen ratio is less than 0.9.

14. A purified protein hydrolysate according to claim 11 comprising additionally fatty and/or flocculated components.

15. Use of the purified protein hydrolysate according to claim 9 as food or fertilizer.

16. Use according to claim 15 as an animal feed additive/supplement, animal feed, fertilizer, or fertilizer concentrate.

17. A food product comprising the purified protein hydrolysate according to claim 9.

18. A fertilizer product comprising the purified protein hydrolysate according to claim 9.
